(19) United States
(12) Patent Application Publication
(10) Pub. No.: US 2023/0009109 A1
(43) Pub. Date: Jan. 12, 2023

(54) OPTIMAL STIMULATION POSITION CALCULATION METHOD, SERVER AND COMPUTER PROGRAM USING AI MODEL

(71) Applicant: NEUROPHET Inc., Seoul (KR)

(72) Inventors: Dong Hyeon KIM, Seoul (KR); Bong Seok KO, Seongnam-si (KR)

(21) Appl. No.: 17/677,884

(22) Filed: Feb. 22, 2022

(30) Foreign Application Priority Data
Jul. 7, 2021 (KR) 10-2021-0089050

(51) Int. Cl. G16H 20/40; G06T 17/20; G06T 7/00; G06V 10/82; G06V 10/764; G06K 9/62; G16H 50/20

(52) U.S. Cl. CPC G16H 20/40; G06T 17/20; G06T 7/0012; G06V 10/82; G06V 10/764; G06K 9/6256; G16H 50/20; G06T 2207/30016; G06T 2207/20084; G06T 2207/20081; G06V 2201/03

(57) ABSTRACT

Provided are optimal stimulation position calculation method, server, and computer program using AI model. The optimal stimulation position calculation method using an AI model, performed on a computing device, include: generating a head shape model based on user diagnosis information; generating a spherical model based on the head shape model; identifying a plurality of transfer coordinate data corresponding to each of a plurality of spherical coordinate data associated with the spherical model; and acquiring optimal stimulation position information by processing the plurality of transfer coordinate data as an input of an optimal position determination model, wherein the plurality of transfer coordinate data are data associated with perpendicular coordinates that can be expressed in the head shape model.

### CROSS-REFERENCE TO RELATED APPLICATION

This application is based on and claims priority under 35 U.S.C. 119 to Korean Patent Application No. 10-2021-0089050, filed on Jul. 7, 2021, in the Korean Intellectual Property Office, the disclosure of which is herein incorporated by reference in its entirety.

### TECHNICAL FIELD

Various embodiments of the present invention relate to an optimal stimulation position calculation method, a server, and a computer program using an AI (Artificial Intelligence) model.

### BACKGROUND ART

Today, various brain stimulation techniques have been used for the treatment of diseases, and in particular, an invasive brain stimulation technique treating diseases by applying electrical signals directly into the brain has been widely used. For example, the invasive brain stimulation technique such as deep brain stimulation (DBS) has been used to treat neurological symptoms such as hand tremor in Parkinson's disease.

Since this invasive brain stimulation technique requires a surgical process in which electrodes are inserted into the brain, it is desirable to perform technique only when it is determined that the pain level the patient is low in comparison of gains and losses and the pain relief effect is clear.

Meanwhile, unlike the invasive brain stimulation techniques which are expensive and risky, recently, non-invasive brain stimulation techniques which are less expensive and less risky have recently been in the spotlight.

Unlike the invasive brain stimulation techniques, the non-invasive brain stimulation techniques do not require a procedure that injures nerve tissue, such as surgery, and thus, the non-invasive brain stimulation techniques guarantee improved safety and do not require hospitalization. In addition, in the non-invasive brain stimulation techniques, since sufficient brain stimulation can be applied only by repeating a single treatment several dozen times in a relatively short period of time, the non-invasive brain stimulation techniques also have o a great advantage in that there is no need to be carried or managed at all times. Representative examples of such non-invasive brain stimulation techniques include transcranial magnetic stimulation (TMS), transcranial direct current stimulation (tDCS), and the like.

It is reported that this non-invasive brain stimulation technique has the ability to enhance or inhibit the activity of brain nerve cells, so that the technique can be not only used for rehabilitation treatment of brain diseases such as cerebral apoplexy but also effective in many brain neurological diseases such as depression, epilepsy, dementia, Parkinson's disease, tic disorders, tinnitus addictions, chronic pains, anxiety disorders, and sleep disorders.

In order to effectively apply stimulation to the brain of the user through the non-invasive brain stimulation technique, it is important to locate the electrodes at the correct positions on the scalp of the user. In other words, in the non-invasive brain stimulation technique, since a specific portion of the brain needs to be stimulated, the accuracy of stimulation is an important factor, so that the electrodes are required to be located at accurate points of the head of the user for the accurate stimulation. In general, as a method of optimizing the electrode positions, there is a method of applying stimulation to various areas on the scalp of the user and measuring the stimulation effect corresponding to the stimulation, but since the electrode test needs to be performed corresponding to each area of the scalp of the patient, the method is not efficient in terms of time and cost. Accordingly, a simulation method exists to optimize the electrode positions. Specifically, as an optimized simulation method for the electrode positions, there is a method of implementing the scalp model, forming many candidate positions in the head while slightly changing the position based on the starting position on the scalp model, and selecting the optimal position through the test at each of the candidate positions.

However, since the human head shape is a non-standardized surface, it may be difficult to continuously change or move the coordinates on the head shape model corresponding to the head shape to select the stimulation position. For example, even if the position of the electrode is slightly moved to the side, since the corresponding position may not be on a scalp but on a skull, the position may not be an appropriate position for arranging the electrodes.

Therefore, in this industrial field, in the process of determining the optimal stimulation position to perform a non-invasive brain stimulation technique, there may be a demand for a modeling method that allows the coordinates to be always located on the scalp even if the coordinates are continuously changed or moved.

### SUMMARY OF INVENTION

### Technical Problem

The purpose of the present invention is to provide an optimal stimulation position calculation method, server, and computer program using an AI model, in which by acquiring a head shape model based on MRI information of each user, acquiring a spherical coordinate system fitted to the corresponding head shape model, and performing electrode model attachment simulation for each of various stimulation positions based on the spherical coordinate system, information on the optimal stimulation position for each user can be acquired.

Effects of the present invention are not limited to the effects mentioned above, and other effects not mentioned will be clearly understood from the following description by those skilled in the art.

### Solution to Problem

According to one embodiment of the present invention, there is provided an optimal stimulation position calculation method using an AI model, performed on a computing device, the method including: generating a head shape model based on user diagnosis information; generating a spherical model based on the head shape model; identifying a plurality of transfer coordinate data corresponding to each of a plurality of spherical coordinate data associated with the spherical model; and acquiring optimal stimulation position information by processing the plurality of transfer coordinate data as an input of an optimal position determination model, wherein the plurality of transfer coordinate data are data associated with perpendicular coordinates that can be expressed in the head shape model.

In various embodiments, the user diagnosis information may be information that is a basis for generating the head shape model and includes medical image information about a brain or head shape of a user, wherein the head shape model is a three-dimensional brain map model associated with a brain or head shape of an actual user, wherein the spherical model is implemented through at least a portion of a three-dimensional sphere model having a specific radius, and wherein the plurality of spherical coordinate data are data associated with spherical coordinates that can be expressed in the spherical model.

In various embodiments, the generating the head shape model based on the user diagnosis information may include: acquiring one or more brain area images by processing the user diagnosis information as an input of an image area classification model; performing pre-processing on the one or more brain area images; and generating the head shape model based on the pre-processed one or more brain area images, and wherein the image area classification model is a neural network model based on a convolutional neural network (CNN), it is to be learned through learning data including learning input data associated with a plurality of the user diagnosis information and learning output data associated with brain area classification information corresponding to each of the user diagnosis information.

In various embodiments, the generating the spherical model based on the user diagnosis information may include: developing one or more spheres based on a center point of the head shape model; identifying a first sphere among the developed one or more spheres having a minimized distance between a surface of a sphere and a surface of the head shape model and having a maximum radius; and generating the spherical model based on the identified first sphere.

In various embodiments, the optimal position determination model may derive brain activity prediction information corresponding to each of the plurality of transfer coordinate data, identify optimal transfer coordinate data in which a loss function associated with a difference between each of the derived brain activity prediction information and each of reference brain activity information corresponding to each of the plurality of transfer coordinate data is smaller than or equal to a preset reference value, and output the optimal stimulation position information based on the identified optimal transfer coordinate data,

In various embodiments, the method may include: determining whether or not at least two or more spherical coordinate data among the plurality of spherical coordinate data indicate one point on the head shape model in an overlapped manner; and performing correction on a portion of the at least two or more spherical coordinate data when the at least two or more spherical coordinate data indicate one point on the head shape model in an overlapped manner.

In various embodiments, the performing the correction on a portion of the at least two or more spherical coordinate data may include removing at least one spherical coordinate data among the at least two or more spherical coordinate data indicating the one point on the head shape model in an overlapped manner; or correcting coordinates of the at least one spherical coordinate data among at least two or more spherical coordinate data indicating the one point on the head shape model in an overlapped manner.

In various embodiments, the method may further include: arranging an electrode model at a first position corresponding to the optimal stimulation position information; and performing electrode attachment simulation on the head shape model by gradually moving the electrode model located at the first position to a position corresponding to the optimal stimulation position information, and wherein the first position is one position in a normal vector direction of the optimal stimulation position information.

According to another embodiment of the present invention, there is provided a server calculating an optimal stimulation position by using an AI model, the server including: a processor; a network interface; a memory; and a computer program loaded on the memory and executed by the processor, and wherein the computer program includes: an instruction for generating a head shape model based on user diagnosis information; an instruction for generating a spherical model based on the head shape model; an instruction for identifying a plurality of transfer coordinate data corresponding to each of the plurality of spherical coordinate data associated with the spherical model; and an instruction for acquiring optimal stimulation position information by processing the plurality of transfer coordinate data as an input of an optimal position determination model, and wherein the plurality of transfer coordinate data are data associated with perpendicular coordinates that can be expressed in the head shape model.

According to another embodiment of the present invention, there is provided a computer program recorded on a computer-readable recording medium, combined with a computing device, and causing the computing device to execute: generating a head shape model based on user diagnosis information; generating a spherical model based on the head shape model; identifying a plurality of transfer coordinate data corresponding to each of a plurality of spherical coordinate data associated with the spherical model, wherein the plurality of transfer coordinate data is data associated with perpendicular coordinates that can be expressed in the head shape model; and acquiring optimal stimulation position information by processing the plurality of transfer coordinate data as an input of an optimal position determination model.

Other specific details of the present invention are included in the detailed description and the drawings.

### Advantageous Effects of Invention

According to various embodiments of the present invention, information on the optimal stimulation position corresponding to each user is provided, so that the treatment efficiency through the non-invasive brain stimulation technique can be improved.

In addition, a model to freely move the coordinates on the scalp is provided, so that convenience can be provided in the process of selecting the optimal stimulation position.

Additionally, simulation of the attachment positions of electrodes of various shapes on a three-dimensional head shape model including a curved surface is provided, and detailed visualization information associated with the electrode arrangement is provided, so that there occurs an effect to maximize the brain stimulation efficiency.

Effects of the present invention are not limited to the effects mentioned above, and other effects not mentioned will be clearly understood from the following description by those skilled in the art.

### BRIEF DESCRIPTION OF DRAWINGS

FIG. 1 is a diagram illustrating an optimal stimulation position calculation system using an AI model according to an embodiment of the present invention.

FIG. 2 is a hardware configuration diagram illustrating a server for calculating an optimal stimulation position using an AI model according to another embodiment of the present invention.

FIG. 3 is a flowchart illustrating an optimal stimulation position calculation method using an AI model according to still another embodiment of the present invention.

FIG. 4 is an exemplary flowchart illustrating a process for generating a head shape model based on user diagnosis information in various embodiments.

FIG. 5 is an exemplary flowchart illustrating a process for determining an optimal position in various embodiments.

FIG. 6 is an exemplary flowchart illustrating a process for performing electrode attachment simulation in various embodiments in various embodiments.

FIG. 7 is an exemplary diagram illustrating a process for acquiring a spherical model based on an MRI image in various embodiments.

FIG. 8 is an exemplary diagram illustrating a spherical model in various embodiments.

FIG. 9 is an exemplary diagram illustrating an electrode model and a spherical model in various embodiments.

FIG. 10 is an exemplary diagram illustrating an electrode arrangement simulation process in various embodiments.

FIG. 11 is an exemplary diagram illustrating a candidate electrode arrangement process in various embodiments.

### DESCRIPTION OF EMBODIMENTS

Advantages and features of the present invention and methods of achieving the advantages and features will become apparent with reference to embodiments described below in detail in association with the accompanying drawings. However, the present invention is not limited to the embodiments disclosed below, but the present invention can be implemented in various different forms, only the embodiments allow the disclosure of the present invention to be complete, the present invention is provided in order for the ordinarily skilled in the art to which the present invention belongs to fully understand the scope of the present invention, and the present invention is only defined by the scope of the claims.

The terminology used herein is for the purpose of describing the embodiments and is not intended to limit the present invention. In this specification, a singular form also includes a plural form unless a phrase specifically states otherwise. As used in this specification, “comprises” and/or “comprising” do not exclude the presence or addition of one or more other components in addition to the stated components. Throughout this specification, the same or similar reference numerals refer to the same or similar elements, and “and/or” includes each and all combination of one or more of the stated elements. Although “first”, “second”, and the like are used to describe various elements, of course, these elements are not limited by these terms. These terms are only used to distinguish one component from other components. Accordingly, it goes without saying that a first component mentioned below may be a second component within the spirit of the present invention.

Unless otherwise defined, all terms (including technical and scientific terms) used herein may be used with the meaning commonly understood by the ordinarily skilled in the art to which the present invention belongs. In addition, terms defined in a commonly used dictionary are not to be interpreted ideally or excessively unless specifically defined explicitly.

As used in this specification, the term “unit” or “module” refers to a software component or a hardware component such as FPGA or ASIC, and the “unit” or “module” performs a certain role. However, the “unit” or “module” is not meant to be limited to software or hardware. The “unit” or “module” may be configured to reside on an addressable storage medium or may be configured to reproduce one or more processors. Accordingly, as an example, the “unit” or “module” includes components such as software components, object-oriented software components, class components and task components, processes, functions, attributes, procedures, subroutines, segments of program codes, drivers, firmware, microcode, circuits, data, database, data structures, tables, arrays, and variables. The components and functions provided within the “unit” or “module” may be combined into a smaller number of components and “units” or “modules” or may be further separated in to additional components and “units” or “modules”.

Spatially relative terms “below”, “beneath”, “lower”, “above”, “upper”, and the like can be used to easily describe the relationship between a certain component and other components. Spatially relative terms should be understood as terms that include different directions of components during use or operation in addition to the directions illustrated in the drawings. For example, in a case where a component illustrated in the drawings is turned over, a component described as “below” or “beneath” of the other component may be placed “above” of the other component. Accordingly, the exemplary term “below” may include both directions below and above. Components may also be oriented in other orientations, and thus, spatially relative terms may be interpreted according to orientation.

In this specification, a computer denotes all types of hardware devices including at least one processor and may be understood as collectively including software configurations operating in a corresponding hardware device according to embodiments. For example, a computer may be understood as meaning including all of a smartphone, a tablet PC, a desktop, a notebook, and a user client and an application running in each device, but the present invention is not limited thereto.

Hereinafter, an embodiment of the present invention will be described in detail with reference to the accompanying drawings.

Each step described in this specification is described as being performed by a computer, but the object of each step is not limited thereto, and at least a portion of each step may be performed in different devices according to embodiments.

Herein, the optimal stimulation position calculation method using the AI model according to various embodiments of the present invention can provide information on the optimal stimulation position in response to each of the plurality of users in the brain stimulation treatment through a non-invasive brain stimulation technique. In addition, the optimal stimulation position calculation method using the AI model of the present invention can generate the head shape model based on the user diagnosis information and can generate and provide the spherical model corresponding to the head shape model. Herein, the spherical model may be a model that allows the coordinates to always be moved on the scalp of the user even in the continuous coordinate change or movement process. That is, the spherical model corresponding to each head shape of the user is provided, so that it is possible to provide convenience in the process of selecting the optimal stimulation position. In addition, the present invention has an effect in that specific visualization information associated with the electrode arrangement is provided by providing simulation for the attachment positions of electrodes with various shapes on the three-dimensional head shape model including a curved surface, so that the brain stimulation efficiency is maximized.

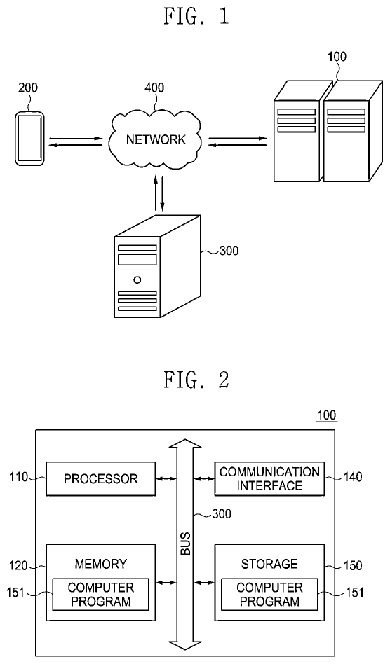

FIG. 1 is a diagram illustrating an optimal stimulation position calculation system using an AI model according to an embodiment of the present invention. As illustrated in FIG. 1, the optimal stimulation position calculation system using the AI model according to an embodiment of the present invention may include an optimal stimulation position calculation server 100, a user terminal 200 and an external server 300.

Herein, the optimal stimulation position calculation system using the AI model illustrated in FIG. 1 is in accordance with one embodiment, and components thereof are not limited to the embodiment illustrated in FIG. 1, but may be added, changed, or deleted as necessary.

In one embodiment, the optimal stimulation position calculation server 100 may generate the head shape model based on the user diagnosis information. For example, the optimal stimulation position calculation server 100 may receive the user diagnosis information of the user. Herein, the user diagnosis information may be information that is the basis for generating the head shape model and may be medical image information about the brain or head shape of the user. For example, the user diagnosis information may be diagnostic information associated with magnetic resonance imaging (MRI). That is, the optimal stimulation position calculation server 100 may generate the head shape model associated with the three-dimensional brain map of the user based on the MRI image of the user.

Specifically, the optimal stimulation position calculation server 100 may acquire one or more brain area images by processing the MRI information of the user as an input of the pre-learned classification model. In this case, the pre-learned classification model may be an image area classification model that classifies one or more brain area images included in the MRI image by setting the MRI image of the user as an input. In other words, when the MRI image of the user is input, the image area classification model may acquire one or more brain area images associated with each of various physical properties of the brain through segmentation of the MRI image.

Herein, the pre-learned image area classification model (for example, an AI model) is configured with one or more network functions, and the one or more network functions are generally configured with a set of interconnected calculation units that can be referred to as ‘nodes’. These ‘nodes’ may also be referred to as ‘neurons’. The one or more network functions are configured with at least one or more nodes. The nodes (or neurons) constituting one or more network functions can be interconnected by one or more ‘links’.

Within the AI model, one or more nodes connected through the link can form a relation between the input node and the output node. The concepts of the input node and the output node are relative ones, and a node in the output node relationship with respect to one node may be in the input node relationship in the relationship with other nodes, and the reverse case is satisfied. As described above, the relationship of the input node to the output node can be generated around the link. One or more output nodes can be connected to one input node through the link, and the reverse case is satisfied.

In the relationship between the input node and the output node connected through one link, the value of the output node can be determined based on the data input to the input node. Herein, the node that interconnects the input node and the output node may have weighted values. The weighted value may be variable, and may be changed by the user or algorithm in order for the AI model to perform a desired function. For example, when the one or more input nodes are interconnected to one output node by the respective links, the output node can determine the output node value based on the values input to the input nodes connected to the output node and the weighted value set in the link corresponding to corresponding to the respective input nodes.

As described above, in the AI model, the one or more nodes are interconnected through the one or more links to form the relationship between an input node and an output node within the AI model. The properties of the AI model can be determined according to the number of nodes and the number of links in the AI model and the correlation between the nodes and the links, and the weighted value allocated to each of the links. For example, when the same number of nodes and the same number of links exist and two AI models having different weighted values between the links exist, the two AI models may be recognized to be different from each other.

Some of the nodes constituting the AI model may constitute one layer based on the distances from the initial input node. For example, a set of nodes having a distance of n from the initial input node may constitute n layers. The distance from the initial input node can be defined by the minimum number of links required to be passed to reach the corresponding node from the initial input node. However, the definition of such a layer is arbitrary for description, and the order of the layer in the AI model may be defined in a manner different from the above. For example, a layer of nodes may be defined by a distance from the last output node.

The initial input node may denote one or more nodes to which data is directly input without going through a link in relation to other nodes among the nodes in the AI model. Alternatively, in the AI model network, in the relationship between nodes based on a link, the above-described nodes may denote nodes not having other input nodes connected by a link. Similarly, the final output node may denote one or more nodes not having an output node in relation to other nodes among the nodes in the AI model. In addition, the hidden node may denote nodes constituting the AI model rather than the first input node and the last output node. The AI model according to one embodiment of the present invention may have more nodes of the input layer than nodes of the hidden layer close to the output layer, and may be an AI model in a form that the number of nodes decreases as the input layer progresses to the hidden layer.

The AI model may include one or more hidden layers. The hidden node of the hidden layer can use the output of the previous layer and the output of the surrounding hidden node as input. The number of hidden nodes for each hidden layer may be the same or different. The number of nodes of the input layer may be determined based on the number of data fields of the input data and may be the same as or different from the number of hidden nodes. The input data input to the input layer may be calculated by a hidden node of the hidden layer and may be output by a fully connected layer (FCL) which is an output layer.

In various embodiments, the AI model may be supervised learning by using the MRI image associated with the plurality of users and the brain area classification information associated with the MRI image as the learning data. However, the present invention is not limited thereto, and various learning methods may be applied.

Herein, the supervised learning is a method of generating the learning data by labeling specific data and information associated with specific data and performing learning by using the learning data and denotes a method of generating the learning data by labeling two data having a causal relationship and performing the learning through the generated learning data.

More specifically, the optimal stimulation position calculation server 100 may generate the image area classification model by performing learning of the one or more network functions constituting the AI model by using the labeled learning data. For example, the optimal stimulation position calculation server 100 can derive the errors by inputting each of the learning input data to one or more network functions and by comparing each of the output data calculated by the one or more network functions with each of the learning output data corresponding to each label of the learning input data. That is, in the learning of the image area classification model, the learning input data may be input to the input layer of the one or more network functions, and the learning output data may be compared with the output of the one or more network functions.

The AI model has been learned by the optimal stimulation position calculation server 100 based on the error between the calculation results of one or more network functions on the learning input data and the learning output data (label).

In addition, the optimal stimulation position calculation server 100 can adjust the weighted values of one or more network functions in a backpropagation method based on the errors. That is, the optimal stimulation position calculation server 100 adjusts the weighed values based on the error between the calculation result of the one or more network functions on the learning output data and the learning input data so that the output of the one or more network functions is close to the learning output data.

In one embodiment, the optimal stimulation position calculation server 100 can determine whether to stop learning by using the validation data when learning of one or more network functions is performed over a preset epoch. The pre-determined epoch may be a portion of the overall learning target epoch.

The validation data may be configured with at least a portion of the labeled learning data. That is, the optimal stimulation position calculation server 100 may perform the learning of the AI model through the learning data, and may determine whether the learning effect of the AI model is above the level determined in advance by using the validation data after the learning of the AI model is repeated for more than the pre-determined epoch. For example, when the optimal stimulation position calculation server 100 performs learning with a target repetition number of 10 using 100 learning data, after performing 10 repetitions of learning which is a pre-determined epoch, 3 repetitions of learning are performed by using 10 validation data. When the change of the AI model output during the 3 repetitions of learning is smaller than or equal to a predetermined level, it may be determined that the more learning is meaningless and the learning may be terminated.

That is, the verification data can be used to determine the completion of learning based on whether the effect of learning for each epoch is above or below a certain level in repeated learning of the AI model. The above-described number of learning data and verification data and the number of repetitions are merely examples, but the present invention is not limited thereto.

The optimal stimulation position calculation server 100 can generate an AI model by testing the performance of one or more network functions by using test data to determine whether one or more network functions are activated. The test data may be used to verify the performance of the AI model, and may be configured with at least a portion of the learning data. For example, 70% of the learning data can be used for training the AI model (that is, learning to adjust the weighted values so as to output the result values similar to the labels), and 30% of the learning data can be used as the test data for verifying the performance of the AI model. The optimal stimulation position calculation server 100 can determine whether or not the AI model is activated according to whether the performance is above a pre-determined performance by inputting the test data into the AI model in which the learning is completed and measuring the error.

When the performance of the AI model in which the learning is completed is verified by using the test data and the performance of the AI model in which the learning is completed is larger than or equal to a predetermined standard, the optimal stimulation position calculation server 100 can activate the AI model to be used in another application.

In addition, when the performance of the AI model in which the learning is completed is smaller than or equal to the pre-determined standard, the optimal stimulation position calculation server 100 may deactivate and discard the AI model. For example, the optimal stimulation position calculation server 100 may determine the performance of the generated AI model based on factors such as accuracy, precision, and recall. The above-described performance evaluation standard is merely examples, and the present invention is not limited thereto. According to one embodiment of the present invention, the optimal stimulation position calculation server 100 can generate a plurality of AI model models by allowing each AI model to be independently learned, and the only AI model with a certain performance or higher in evaluation of the performance can be used. However, the present invention is not limited thereto.

Throughout this specification, a calculation model, a neural network, a network function, and a neural network may be used to have the same meaning. (Hereinafter, the neural networks will be unified and described) the data structure may include neural networks. And the data structure including the neural network may be stored in a computer readable medium. The data structures including the neural networks may also include data input to the neural network, weighted values of the neural network, hyper-parameters of the neural network, data acquired from the neural network, activation functions associated with each node or layer of the neural network, and loss functions for learning the neural network. The data structure including the neural network may include arbitrary the components among the disclosed configuration. That is, the data structure including the neural network may be configured to include all or arbitrary combinations of the data input to the neural network, the weighted values of the neural network, the hyper-parameters of the neural network, the data acquired from the neural network, the activation functions associated with each node or layer of the neural network, the loss functions for learning the neural network, and the like. In addition to the above-described configurations, the data structure including the neural network may include any other information for determining the properties of the neural network. In addition, the data structure may include all types of data used or generated in the calculation process of the neural network, but the present invention is not limited to the above. The computer-readable medium may include a computer-readable recording medium and/or a computer-readable transmission medium. The neural network can be configured as a set of interconnected calculation units that can be generally referred to as nodes. The nodes may also be referred to as neurons. The neural network is configured with at least one or more nodes.

In various embodiments, the optimal stimulation position calculation server 100 can be connected to the user terminal 200 through the network 400, and can generate and provide the head shape model associated with the three-dimensional brain map corresponding to the MRI image by using the AI model, and in addition, the information on the optimal stimulation position for transcranial direct current stimulation (tDCS) in the actual head shape of the user can be provided based on the corresponding head shape model. According to one embodiment, the optimal stimulation position calculation server 100 may be a server that provides information on the brain stimulation position in relation to a single channel using two electrodes.

In addition, herein, the network 400 may have a connection structure capable of exchanging information between respective nodes such as a plurality of terminals and servers. For example, the network 400 includes a local area network (LAN), a wide area network (WAN), the Internet (WWW: World Wide Web), a wired/wireless data communication network, a telephone network, a wired/wireless television communication network, or the like.

In addition, herein, the wireless data communication network includes 3G, 4G, 5G, a third generation partnership project (3GPP), a fifth generation partnership project (5GPP), a long term evolution (LTE), a world interoperability for microwave access (WIMAX), a Wi-Fi, the Internet, a local area network (LAN), a wireless local area network (Wireless LAN), a wide area network (WAN), a personal area network (PAN), a radio frequency (RF), a Bluetooth network, a near-field communication (NFC) network, a satellite broadcasting network, an analog broadcasting network, a digital multimedia broadcasting (DMB) network, or the like, but the wireless data communication network is not limited thereto.

In one embodiment, the user terminal 200 may be connected to the optimal stimulation position calculation server 100 through the network 400, may provide the user diagnosis information (for example, the MRI image) to the optimal stimulation position calculation server 100, and may be provided with various types of information (for example, a head shape model associated with a three-dimensional brain map, the information on the optimal stimulation position for tDCS treatment, or the like) as a response to the provided user diagnosis information.

Herein, the user terminal 200 is a wireless communication device that guarantees portability and mobility and includes all types of handheld-based wireless communication device such as a navigation, a personal communication system (PCS), a global system for mobile communications (GSM), a personal digital cellular (PDC), a personal handyphone system (PHS), a personal digital assistant (PDA), an international mobile telecommunication (IMT)-2000, a code division multiple access (CDMA)-2000, a W-code division multiple access (W-CDMA), a wireless broadband internet (Wibro) terminal, a smartphone, a smart pad (Smartpad), and a tablet PC, but the user terminal is not limited thereto.

In one embodiment, the user terminal 200 may be a terminal associated with a user accessing the server 100 to acquire an optimal stimulation position based on the user diagnosis information (for example, the MRI image). The user terminal 200 may transmit the MRI image photographed or stored in the user terminal 200 to the server 100. The user terminal 200 may be, for example, a terminal associated with a specialist providing a transcranial direct current stimulation procedure to a patient based on MRI information. When the user terminal 200 is a terminal associated with a specialist providing transcranial direct current stimulation, the optimal stimulation position information received from the server 100 may be used as medical assistance information for clear stimulation position determination. The user terminal 200 has a display and can receive an input of the user and provide an output of any type to the user. In one embodiment, the user of the user terminal 200 may be a medical professional and may denote a doctor, a nurse, a clinical pathologist, a medical image expert, or the like, and may be a technician repairing a medical device, but the present invention is not limited thereto.

In one embodiment, the external server 300 may be connected to the optimal stimulation position calculation server 100 through the network 400, and may provide information/data required for the optimal stimulation position calculation server 100 to perform the method of calculating the optimal stimulation position by using the AI model or may be provided with, store, and manage the result data derived as performing the method of calculating the optimal stimulation position by using the AI model. For example, the external server 300 may be a storage server separately provided outside the server 100, but the external server is not limited thereto. Hereinafter, the hardware configuration of the server 100 performing the optimal stimulation position calculation method using the AI model will be described with reference to FIG. 2.

FIG. 2 is a hardware configuration diagram illustrating an optimal stimulation position calculation server using the AI model according to another embodiment of the present invention.

Referring to FIG. 2, an optimal stimulation position calculation server 100 (hereinafter, a “server 100”) according to another embodiment of the present invention may include one or more processors 110, a memory 120 on which a computer program 151 executed by the processor 110 is loaded, a bus 130, a communication interface 140, and a storage 150 which stores the computer program 151. Herein, only components associated with the embodiment of the present invention are illustrated in FIG. 2. Accordingly, a person skilled in the art to which the present invention belongs can know that other general-purpose components other than the components illustrated in FIG. 2 may be further included.

The processor 110 controls the overall operations of each configuration of the server 100. The processor 110 may be configured to include a central processing unit (CPU), a micro process or unit (MPU), a micro controller unit (MCU), a graphic processing unit (GPU), or any type of processors well known in the art of the present invention.

The processor 110 may read the computer program stored in the memory 120 and perform data processing for the AI model according to one embodiment of the present invention. According to an embodiment of the present invention, the processor 110 may perform an operation for learning a neural network. The processor 110 may perform calculations for learning of the neural network such as processing input data for learning in deep learning (DL), extracting features from the input data, calculating errors, and updating weighted values of the neural network by using back-propagation.

In addition, the processor 110 may allow at least one of the CPU, the GPGPU, and the TPU to process the learning of the network function. For example, the CPU and the GPGPU can process the learning of the network function and the data classification using the network function. In addition, in one embodiment of the present invention, the learning of the network function and the data classification using the network function may be processed by using processors of a plurality of computing devices together. In addition, the computer program performed in the computing device according to an embodiment of the present invention may be a CPU, GPGPU, or TPU executable program.

In this specification, the network function can be used interchangeably with artificial neural networks and neural networks. In this specification, the network function may include one or more neural networks, and in this case, an output of the network function may be an ensemble of outputs of one or more neural networks.

The processor 110 may read the computer program stored in the memory 120 and provide the image area classification model according to one embodiment of the present invention. According to one embodiment of the present disclosure, the processor 110 may classify the image as one or more brain area images. According to an embodiment of the present invention, the processor 110 may perform calculation for allowing the image area classification model to be learned.

According to one embodiment of the present invention, the processor 110 may typically process the overall operations of the server 100. The processor 110 may provide or process appropriate information or functions to the user or the user terminal by processing signals, data, information, or the like input or output through the above-described components or driving an application program stored in the memory 120.

In addition, the processor 110 may perform an operation for at least one application or program for executing the method according to embodiments of the present invention, and the server 100 may include the one or more processors.

In various embodiments, the processor 110 may further include a random access memory (RAM, not illustrated) and a read only memory (ROM, not illustrated) temporarily and/or permanently storing signals (or data) processed inside the processor 110. In addition, the processor 110 may be implemented in a form of a system on chip (SoC) including at least one among a graphic processing unit, a RAM, and a ROM.

The memory 120 stores various data, commands and/or information. The computer program 151 from the storage 150 in order to perform methods/operations according to various embodiments of the present invention is loaded on the memory 120. When the computer program 151 is loaded on the memory 120, the processor 110 may perform the method/operation by performing one or more instructions constituting the computer program 151. The memory 120 may be implemented as a volatile memory such as RAM, but the technical scope of the present disclosure is not limited thereto.

The bus 130 provides a communication function between the components of the server 100. The bus 130 may be implemented as various types of buses such as an address bus, a data bus, and a control bus.

The communication interface 140 supports wired/wireless Internet communication of the server 100. In addition, the communication interface 140 may support various communication methods other than the Internet communication. For this supporting, the communication interface 140 may be configured to include a communication module well known in the technical field of the present invention. In some embodiments, the communication interface 140 may be omitted.

The storage 150 may non-temporarily store the computer program 151. When the optimal stimulation position calculation process using the AI model is performed through the server 100, the storage 150 may store various types of information necessary to provide the optimal stimulation position calculation process using the AI model.

The storage 150 may be configured to include a non-volatile memory such as a read only memory (ROM), an erasable programmable ROM (EPROM), an electrically erasable programmable ROM (EEPROM), or a flash memory, a hard disk, a removable disk, a computer-readable recording medium of an arbitrary form well-known in the technical field to which the present invention belongs.

The computer program 151 may include one or more instructions that, when the computer program is loaded on the memory 120, the processor 110 is allowed to perform a method/operation according to various embodiments of the present invention. That is, the processor 110 may perform the method/operation according to various embodiments of the present invention by executing the one or more instructions.

In one embodiment, the computer program 151 includes one or more instruction for executing an optimal stimulation position calculation method using an AI model, including: a step of generating a head shape model based on user diagnosis information; a step of generating a spherical model based on the head shape model; a step of identifying a plurality of transfer coordinate data corresponding to each of a plurality of spherical coordinate data associated with the spherical model; and a step of acquiring optimal stimulation position information by processing the plurality of transfer coordinate data as an input of an optimal position determination model.

The steps of the method or algorithm described in relation to the embodiment of the present invention may be implemented directly in hardware, implemented as a software module performed by hardware, or implemented by a combination thereof. The software module may reside in a random access memory (RAM), a read only memory (ROM), an erasable programmable ROM (EPROM), an electrically erasable programmable ROM (EEPROM), a flash memory, a hard disk, a detachable disk, a CD-ROM, or an arbitrary type of a computer-readable recording medium well known in the art to which the present invention belongs.

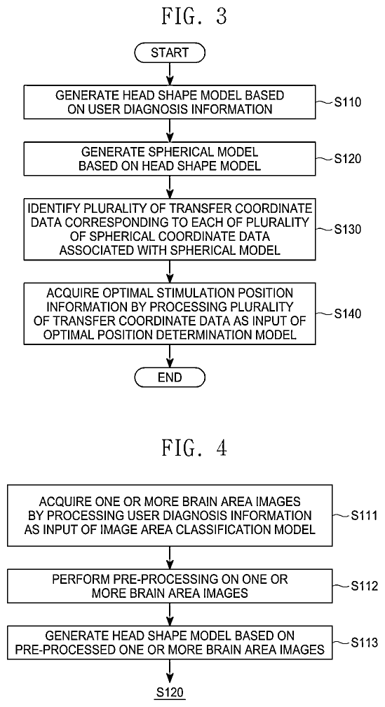

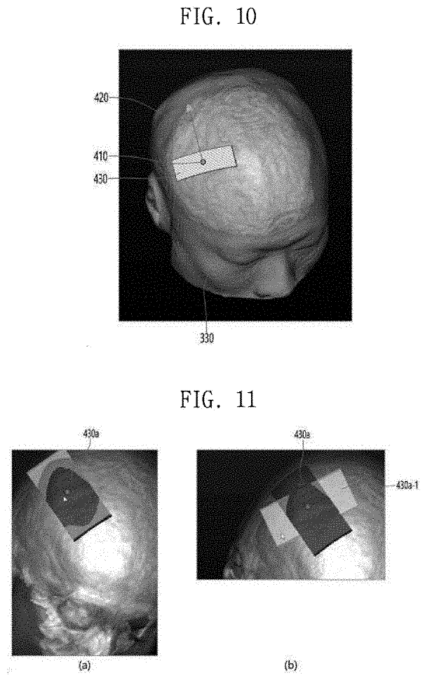

The components of the present invention may be implemented as a program (or application) to be performed in combination with a computer that is hardware and stored in a medium. The components of the present invention may be implemented as software programming or software components, and similarly, in the embodiments, various algorithms implemented as data structures, processes, routines, or combinations of other programming components are included, and the algorithms can be implemented in a programming or scripting language such as C, C++, Java, or assembler. Functional aspects can be implemented as algorithms performed by one or more processors. Hereinafter, the optimal stimulation position calculation method using the AI model performed by the server 100 will be described with reference to FIGS. 3 to 11.

FIG. 3 is a flowchart illustrating the optimal stimulation position calculation method using the AI model according to another embodiment of the present invention.

In one embodiment, the server 100 may acquire the user diagnosis information. Herein, the user diagnosis information is information that becomes the basis for generating the head shape model 330 and may include medical image information about the brain or head shape of the user. For example, the user diagnosis information may be a magnetic resonance image (that is, an MRI image) obtained by photographing the head including the brain of the object. That is, the user diagnosis information in the present invention may refer to the MRI images including not only the brain but also the skull and scalp of the object.

According to an embodiment of the present invention, the acquisition of the user diagnosis information may be receiving or loading the user diagnosis information stored in the memory 120. In addition, the acquisition of the user diagnosis information may be receiving or loading data from another storage medium, another server, or a separate processing module within the same server based on wired/wireless communication means.

Referring to FIG. 3, in step S110, the server 100 may generate the head shape model 330 based on the user diagnosis information. Herein, the head shape model may denote the three-dimensional brain map model associated with the brain or head shape of actual users. In other words, the server 100 may generate the head shape model 330 associated with the three-dimensional brain map model for the user based on the MRI image including the brain, skull, and scalp of the user.

Referring to FIG. 4, in step S111, the server 100 may acquire one or more brain area images 320 by processing the user diagnosis information as an input of the image area classification model. Herein, the image area classification model may be a CNN-based neural network model learned through the learning data including learning input data associated with a plurality of the user diagnosis information and learning output data associated with the brain area classification information corresponding to each of the user diagnosis information.

As a specific example, the server 100 can generate the learning data by using the MRI image 310 as the learning input data, by using one or more brain area (that is, correct answer) images included in the MRI image 310 as the learning output data, and by labeling the learning input data and the learning output data. The server 100 may process the learning input data as an input of one or more network functions and output learning result data. In this case, result data may be information about one or more brain area images. The server 100 can derive an error between the learning result data output from the one or more network functions and the learning output data labeled in the learning input data. The server 100 can perform learning for one or more network functions by back-propagating the error and adjusting the weighted values of the neural network so that the learning result data is similar to the learning output data. As described above, as the learning of one or more network functions is completed, an image area classification model may be generated. That is, the image area classification model can classify one or more brain area images from the MRI image of the user by the above-described learning. Herein, each of the one or more brain area images may be associated with each of various physical properties (for example, a white matter, a gray matter, a brain spinal fluid, a skull, a scalp, or the like) of the brain.

As an embodiment, the image area classification model may include one or more batch normalization layers, activation layers, and convolution layers, but the present invention is not limited thereto.

In addition, in one embodiment, the image area classification model may be configured to include a horizontal pipeline configured with a plurality of blocks for extracting high-level properties from low-level properties of the MRI image and a vertical pipeline for performing the segmentation by collecting the properties extracted from the horizontal pipelines so as to perform the segmentation on the MRI image with poor image quality.

In addition, in step S112, the server 100 may perform pre-processing for one or more brain area images 320. The pre-processing performed by the server 100 may denote pre-processing of removing noise from each brain area image. In one embodiment, the server 100 may perform pre-processing associated with noise rejection and pre-processing associated with hole removal based on the maximum chunk.

In one embodiment, the server 100 may perform pre-processing of noise rejection based on the maximum chunk. Specifically, the server 100 may perform pre-processing of removing the remaining chunks except for the largest chunk (that is, the largest connected component) in each brain area image. For example, as a result of the segmentation of the image area classification model, when several components exist in each image, it may denote that the accuracy of the classification is low. When the head shape model, which is a three-dimensional mesh model, is generated based on the images with a low-accuracy of classification, there is a concern that the accuracy of the generated head shape model is somewhat lacking. That is, the server 100 can improve the accuracy of segmentation classification by performing pre-processing that removes the remaining small components except for the largest component on the image through noise rejection pre-processing based on the maximum chunk.

In addition, the server 100 may perform pre-processing associated with the hole removal. Specifically, the server 100 may perform pre-processing of removing holes in each brain area image, that is, filling in the hole portions. For example, the holes existing on each brain area image may be errors generated by a CNN-based segmentation process. Accordingly, the server 100 has the effect of being capable of improving the accuracy of segmentation classification by performing the pre-processing of removing the holes which are kinds of segmentation errors and contributing to the improvement of the accuracy of generation of the head shape model through the brain area images classified later.

In addition, in step S113, the server 100 may generate a head shape model 330 based on the pre-processed one or more brain area images 320. The server 100 may generate a three-dimensional brain map based on the properties of each of the brain area images classified through the image area classification model. That is, the server 100 may generate the three-dimensional head shape model 330 configured with a plurality of meshes based on each brain area image.

In one embodiment, the server 100 may acquire information about physical properties associated with each brain area. Herein, the physical properties associated with each brain area may include at least one of isotropic and anisotropic electrical conductivities. For example, the isotropic electrical conductivity may be acquired by allocating the electrical conductivity known through an experiment to each segmented area.

According to another embodiment of the present invention, the server 100 may perform the simulation of stimulation application for applying stimulation to the head shape model 330 associated with the three-dimensional brain map. The simulation of stimulation application is to simulate a transcranial direct current stimulation technique of arranging the electrode model 430 on one side of the head of the user, applying the electrical stimulation to the user through the electrode model 430 to stimulate a specific portion of the brain for the treatment. For example, when a specific electrical stimulation is applied to one point on the head, a state in which the specific electrical stimulation propagates in the brain of an object can be simulated.

Specifically, the server 100 may simulate a state in which the electrical stimulation propagates in the brain of the user by using the head shape model 330 and the physical properties constituting the corresponding head shape model 330. For example, the electrical stimulation that may be applied to the head of the user may include at least one of a magnetic field, an electric field, and an electric current. For example, when a magnetic field is applied to the head of the user, the current induced by the magnetic field may be propagated in the brain of the user.

According to one embodiment, the server 100 may acquire an optimal position for applying the electrical stimulation based on the head shape model 330 of the user. For example, the server 100 can check several paths for the electrical stimulation to be transmitted from the scalp of the user to the stimulation target point through the head shape model 330 and can search for an optimal position to apply the electrical stimulation to the head among the paths. Herein, the optimal position where the stimulation is to applied may be one point on the scalp that maximizes the electrical stimulation applied to the stimulation target point through the electrode model 430.

The server 100 can perform the simulation of stimulation application while gradually changing the coordinates by using one position as the reference point to search for the optimal position to be applied with the stimulation. As a result of performing the simulations in various positions, one point on the scalp where the electrical stimulation applied to the stimulation target point is maximized can be determined as the optimal stimulation position.

However, in the process of performing the above procedure, when the coordinates are slightly changed using one position (that is, coordinate) as the reference point, as the head shape model 330 is a non-standardized surface (that is, the head shape of the specific user which becomes the basis for generation of the head shape model is a non-standardized surface), it may be difficult to continuously move the correct coordinates. Specifically, it may be difficult to move on the scalp of the head shape model for the user through changes based on the variables of the three-dimensional coordinate, that is, the x, y, and z axes, and thus, errors may occur in the process of performing the simulation of stimulation application while gradually changing the coordinate.

As a specific example, in the three-dimensional coordinate system, when the electrode is moved by a certain distance along the x-axis to change the position in which the electrode is arranged, the position may be an area inside the skull rather than an area on the scalp.

There is a concern in that this may cause the errors in determining the optimal stimulation position through the simulation of stimulation application, and as a result, the errors may reduce the brain disease treatment efficiency through the transcranial direct current stimulation.

Accordingly, the server 100 of the present invention may generate a spherical model 340 that allows the coordinates to always be located on the scalp even when the coordinates are continuously changed or moved based on the three-dimensional head shape model 330 of the user. That is, the server 100 generates the spherical model 340 corresponding to the head shape model 330 of the user and sequentially moves the test positions based on the spherical coordinate data that can be expressed in the corresponding spherical model 340, so that it is possible to search for the optimal position where the stimulation is to be applied. In other words, the server 100 can enable continuous coordinate transformation in the process of simulation of stimulation application to search for the optimal stimulation position through the generation of the spherical model 340.

More specifically, as in step S120, the server 100 may generate the spherical model 340 based on the head shape model 330. The spherical model 340 may be implemented through at least a portion of the three-dimensional sphere model having a specific radius. The spherical model 340 may be a model that can be expressed through a plurality of the spherical coordinate data. The plurality of spherical coordinate data may be data associated with spherical coordinates regarding theta and pi that can be expressed in the spherical model.

Specifically, the server 100 may develop one or more spheres based on the center point of the head shape model 330. The developing one or more spheres may denote developing spheres with different radii based on an arbitrary center point. In addition, the server 100 can identify the first sphere with the largest radius minimizing the distance between the surface of the sphere among the developed one or more spheres and the surface of the head shape model 330. In other words, the server 100 develops a sphere having various radii based on an arbitrary center point of the head shape model 330, and identifies the first sphere minimizing the distance from the surface of the head shape model 330 among the developed spheres and having a maximum radius.

In one embodiment, the minimizing of the distance between the surface of the sphere and the surface of the head shape model 330 may denote that the average of the distance difference between all points on the surface of the head shape model 330 and the corresponding all points on the surface of the sphere is minimized. That is, the first sphere may denote a sphere where a difference, which is a root mean square error (RMSE) of an average distance from one point associated with the surface (for example, points corresponding to the upper hemisphere or points corresponding to the entire area) of the head shape model 330 is minimized and a radius is maximized. In addition, the server 100 may generate the spherical model 340 based on the identified first sphere.

According to an additional embodiment, the server 100 may identify a sphere with a radius tangent to the inside of the first point of the head shape model 330. A point at which the radius is maximized based on the virtual center point on the head shape model 330 may be a characteristic. The server 100 may generate the spherical model 340 based on the identified sphere. That is, the server 100 may generate the spherical model 340 based on a sphere with a maximum radius while being in contact with a portion of the surface of the head shape model 330.

In other words, the server 100 may identify a sphere that can surrounds the head shape model 330 and may generate, based on the radius of the sphere, the spherical model 340 where the coordinates are always located on the scalp even when the coordinates are continuously changed or moved. That is, the server 100 may generate the spherical model 340 based on the head shape model 330 of the user by using the head shape of the user and the sphere with the smallest RMS error. Accordingly, the spherical model 340 may be implemented through at least a portion of a sphere that is most closely fit to the head of the user.

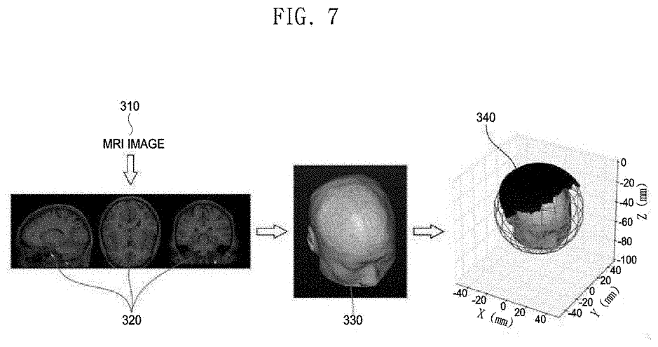

In other words, as illustrated in FIG. 7, when the server 100 receives the MRI image 310 of the user, the server 100 can acquire the one or more brain area images 320 based on the corresponding MRI image 310, can generate the head shape model 330 based on the corresponding one or more brain area images 320, and can generate the spherical model 340 based on the generated head shape model 330.

That is, the spherical model 340 generated through the above-described process is implemented based on the three-dimensional head shape model 330 generated in response to the MRI image of the user and surrounds the head shape model 330. According to the implementation, when the corresponding head shape model 330 is used, coordinate data that is movable on the scalp of the user, that is, on the scalp of the head shape model 330 can be identified.

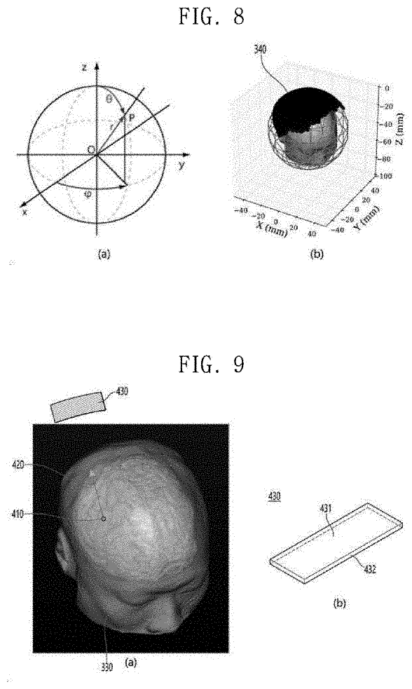

In other words, since the spherical model 340 as illustrated in FIG. 8 is generated, continuous coordinate transformation can be performed on the scalp of the user and the simulation for the optimal stimulation position calculation can be performed. In addition, when the spherical model 340 is used, as the existing coordinate variables are converted from three variables (for example, x, y, z) to two variables (θ, ϕ), convenience can be improved in this process of continuously transforming the coordinates for the simulation.

In addition, in step S130, the server 100 may identify the plurality of transfer coordinate data corresponding to each of the plurality of spherical coordinate data associated with the spherical model 340. Herein, the plurality of transfer coordinate data may be coordinates on the actual head shape model 330 transferred to each of the plurality of spherical coordinates. That is, the plurality of transfer coordinate data may be data associated with perpendicular coordinates that can be expressed on the head shape model 330. The server 100 may identify the spherical coordinate data where the user can move on the scalp even after continuous coordinate transformation through the spherical model 340 and may identify the transfer coordinate data associated with the coordinate transferred to the head shape model 330 based on the spherical coordinate data.

In step S140, the server 100 may acquire the optimal stimulation position information by processing the plurality of transfer coordinate data as an input of the optimal position determination model. In other words, the server 100 identifies the spherical coordinate data that can be moved on the scalp of the user even in continuous coordinate transformation through the spherical model 340, and optimal stimulation position information can be acquired based on a plurality of transfer data corresponding to the identified spherical coordinate data. That is, in the present invention, the optimal stimulation position is not determined as a point where the stimulation position is maximum based on continuous coordinate transformation in the head shape model 330, but is determined based on coordinate transformation in the spherical model 340 corresponding to the head shape model 330. The optimal stimulation position information may be information on the optimal position to which the electrical stimulation is to be applied from the scalp of the user to the stimulation target point as a result of the simulation. For example, the optimal position to be applied with the electrical stimulation may be one point on the scalp so as to maximize the electrical stimulation applied to the stimulation target point through the electrode model 430.

That is, the server 100 may acquire the optimal stimulation position information based on the plurality of transfer coordinate data corresponding to the plurality of spherical coordinate data identified through the spherical model 340. The server 100 may acquire the optimal stimulation position information by processing the plurality of transfer coordinate data as an input of the optimal position determination model.

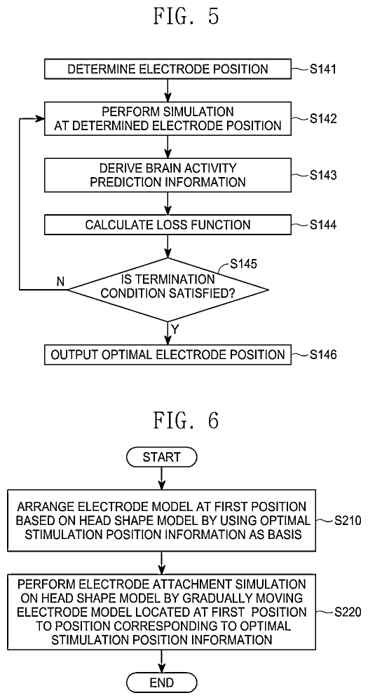

Specifically, the optimal position determination model may be a model designed to perform an optimization algorithm that outputs the optimal stimulation position information by using the plurality of transfer coordinate data as input. The optimal position determination model of the present invention may derive brain activity prediction information corresponding to each of the plurality of transfer coordinate data, may identify the optimal transfer coordinate data where the loss function associated with the difference between each of the derived brain activity prediction information and each of the reference brain activity corresponding to each of the plurality of transfer coordinate data is smaller than or equal to the preset reference value, and may output the optimal stimulation position information based on the identified optimal transfer coordinate data. More specifically, the optimization algorithm performed by the optimal position determination model may be as illustrated in FIG. 5.

More specifically described with reference to FIG. 5, the optimal position determination model may determine the electrode position based on at least one of the plurality of transfer coordinate data (S141). Herein, the electrode position determination may be associated with the initial electrode position which is an area on the scalp for arranging the electrode model 430.

In the optimal position determination model, when the initial electrode position is determined, the simulation can be performed with the determined electrode position. Specifically, the optimal position determination model can perform the simulation of stimulation application to apply the stimulation to the head shape model 330 associated with the three-dimensional brain map based on the determined initial electrode position (S142). For example, when the electrical stimulation is applied to the corresponding electrode position, a state in which a specific electrical stimulation propagates in the brain can be simulated.

In addition, the optimal position determination model can derive the brain activity prediction information according to the result of the above-described simulation (S143). Herein, the brain activity prediction information may include a value derived as a result of the simulation where the electrical stimulation is applied to the determined electrode position, for example, a value associated with an electric field. That is, the brain activity prediction information may be prediction information associated with the electric field value generated at a specific point of the brain as the electrical stimulation is applied to the determined electrode position. For example, the greater the value of the brain activity prediction information generated in response to the electrical stimulation, the closer to the optimal stimulation position.

In addition, the optimal position determination model can calculate the loss function associated with the difference between the brain activity prediction information and the reference brain activity information. Herein, the reference brain activity information is associated with the electric field value at a desired point and may be, for example, a value arbitrarily set by the user.

In addition, the optimal position determination model may determine whether or not the termination condition is satisfied based on the loss function associated with the difference between the derived brain activity prediction information corresponding to the determined point and the reference brain activity information (S145). The optimal position determination model may determine whether or not the termination condition is satisfied based on whether or not the loss function is smaller than or equal to a preset reference value.

More specifically, when the loss function exceeds the preset reference value, as the difference between the brain activity prediction information and the reference brain activity information is large, the optimal position determination model determines that the termination condition is not satisfied and searches for a new electrode position. For example, the optimal position determination model may move the spherical coordinate data on the spherical model 340 based on the spherical coordinate data associated with the initial position and may determine the new electrode position based on the transfer coordinate data corresponding to the moved spherical coordinate data.

In addition, when the loss function is smaller than or equal to a preset reference value, the optimal position determination model may determine that the termination condition is satisfied and determine the corresponding position as the optimal electrode position (S146).

That is, as described above, as the optimization algorithm is repeatedly performed in such a way that the loss function is minimized, the optimal stimulation position information associated with the position at which the optimal stimulation is expected to be applied among the plurality of transfer coordinate data can be acquired.

As a result, since the optimal stimulation position information with improved accuracy can be acquired through the simulation of stimulation application. when the transcranial direct current stimulation technique is to be used for the brain disease treatment, there occurs an effect to provide customized guide such as assisting medical procedures by providing high-quality information corresponding to each head shape for each of the various users, and as a result, the efficiency of the brain disease treatment can be improved.

According to one embodiment of the present invention, the server 100 may determine whether or not at least two or more spherical coordinate data among the plurality of spherical coordinate data indicate one point of the head shape model 330 in an overlapped manner. For example, the spherical coordinate data may be coordinate data that can be expressed in the spherical model 340 corresponding to the head shape model 330 so that the coordinate can move on the head shape of the user even with continuous coordinate transformation. That is, the spherical coordinate data may be the spherical coordinate data included in the spherical model 340 formed to surround the head model 330 inward, and may be displayed based on the spherical coordinate system.

Since the plurality of spherical coordinate data is based on the spherical model 340, it can always be moved only on the scalp of the user, but when the electrode is to be attached based on the spherical coordinate data, one point indicated by each spherical coordinate data on the head shape of the user may overlap each other. In other words, the points may be different on the spherical coordinate system, but the positions transferred on the scalp may overlap.

When different points on the spherical coordinate system overlap after being transferred onto the scalp, a decrease in efficiency in the optimal stimulation position search process may be caused. As a specific example, it is possible to determine whether or not the termination condition is satisfied based on the loss function at the first position. When the termination condition is not satisfied, the second position which is another point (or position) on the head shape is searched for, and the simulation of stimulation application can be performed at the second position. In this case, the first position and the second position may be different points on the spherical coordinate system, but the positions transferred on the scalp may overlap. As a result, since the simulation of stimulation application associated with the same point is performed again, the efficiency of the simulation to acquire the optimal stimulation position may be reduced.

Accordingly, the server 100 according to an embodiment of the present invention may determine whether or not at least two or more spherical coordinate data among the plurality of coordinate data indicate one point of the head shape model 330 in an overlapped manner and may correct at least a portion of the spherical coordinate data determined to indicate one point in an overlapped manner.

In more detail, the server 100 determines whether or not at least two or more spherical coordinate data among the plurality of spherical coordinate data indicate one point of the head shape model 330 in an overlapped manner based on the plurality of transfer coordinate data. The plurality of transfer coordinate data may be coordinates on the actual head shape model 330 transferred from each of the plurality of spherical coordinates. The plurality of transfer coordinate data may be data associated with perpendicular coordinates that can be expressed in the head shape model 330. That is, when at least one transfer coordinate data among the plurality of transfer coordinate data transferred to the head shape model 330 based on each of the plurality of spherical coordinate data is matched with the two or more spherical coordinate data, the server 100 can determined that at least two or more spherical coordinate data indicate the same one point of the head shape model 330.

That is, as described above, when different points on the spherical coordinate system or the positions transferred on the scalp overlap, the server 100 may determines that at least two or more spherical coordinate data indicate one point of the head shape model 330 in an overlapped manner.

According to one embodiment of the present invention, when the at least two or more spherical coordinate data indicate one point of the head shape model 330 in an overlapped manner, the server may perform the correction on a portion of the at least two or more spherical coordinate data.

Specifically, the server 100 may remove at least one spherical coordinate data among at least two or more spherical coordinate data that indicate one point of the head shape model 330 in an overlapped manner. For example, when the spherical coordinate data corresponding to the first transfer coordinate data is the first spherical coordinate data and the second spherical coordinate data, the server 100 may remove the first spherical coordinate data. The specific description of the transfer coordinate data and spherical coordinate data described above is merely an example, and the present disclosure is not limited thereto.

That is, the server 100 removes at least one of the spherical coordinate data corresponding to the same point on the head shape model 330, so that only the simulation corresponding to one spherical coordinate data can be allowed to be performed in the simulation process of acquiring the optimal stimulation position, and the overall simulation efficiency can be maximized.

According to another embodiment, the server 100 may correct the coordinates of at least one spherical coordinate data among at least two or more spherical coordinate data that indicate one point of the head shape model 330 in an overlapped manner. For example, when the spherical coordinate data corresponding to the first transfer coordinate data is the first spherical coordinate data and the second spherical coordinate data, the server 100 may generate the spherical coordinate data by adjusting the theta or pi of the first spherical coordinate data. The specific description of the transfer coordinate data and spherical coordinate data described above is merely an example, and the present disclosure is not limited thereto.

That is, the server 100 can maximize the overall simulation efficiency by adjusting at least one of spherical coordinate data corresponding to the same point on the head shape model 330 to correct the coordinates so that overlapped positions do not occur in the simulation process of acquiring the optimal stimulation position.

According to one embodiment of the present invention, the server 100 may perform the electrode attachment simulation to attach the electrode model 430 to the head shape model 330. The electrode attachment simulation may be to provide the information on the process in which the electrode model 430 is contacted or attached to the head shape model 330. For example, visualization information associated with how to attach the electrode patch to the actual scalp of the user can be provided through the electrode attachment simulation.

As illustrated in FIG. 9, the electrode model 430 of the present invention is a three-dimensional model obtained by modeling an electrode patch attached to the scalp of an actual user and may include a first surface 432 that can be in contact with the head shape model 330 and a second surface 431 corresponding to the first surface 432. In one embodiment, the coordinates forming each of the first surface 432 and the second surface 431 may have the same directivity. That is, when the first surface 432 of the electrode model 430 is moved in one direction in the simulation process, the second surface 431 may also be moved in the same direction.

Specifically, as illustrated in FIG. 6, in step S210, the server 100 may arrange the electrode model at a first position corresponding to the optimal stimulation position information. Herein, the first position may be one position in the direction of the normal vector 410 of the optimal stimulation position information. That is, the server 100 can arrange the electrode model 430 at the first position in the direction of the normal vector 410 outside the head shape from the one point on the scalp where the optimal stimulation is predicted to be applied. In other words, the electrode model 430 may be located at one point (that is, the first position) separated from the one point (that is, the optimal stimulation position), which is to be a target, in the normal vector direction.

In addition, in step S220, the server 100 may perform the electrode attachment simulation for the head shape model 330 by gradually moving the electrode model 430 located in the first position to the position corresponding to the optimal stimulation position information.

More specifically, when the first surface 432 of the electrode model 430 and the head shape model 330 are in contact with each other, the server 100 may stop the movement of the corresponding electrode model 430. In other words, when the first surface 432 of the electrode model 430 is in contact with the one point (for example, the optimal stimulation position) of the head shape model 330, the movement of the electrode model 430 may be stopped.

In addition, the server 100 may calculate the movement distance of each of the plurality of first coordinates in relation to the first surface 432 of the electrode model 430 where the movement is stopped. For example, as the electrode model 430 is moved from the first position to one point of the head shape model, the plurality of first coordinates constituting the first surface (that is, the bottom surface) of the corresponding electrode model 430 may be changed. The server 100 can calculate the movement distance of the first coordinates.

In addition, the server 100 may move each of the plurality of second coordinates in association with the second surface 431 based on the movement distance of each of the plurality of first coordinates. For example, as the electrode model 430 is moved from the first position to the one point of the head shape model 330, when the plurality of first coordinates constituting the first surface 432 of the electrode model 430 are moved by ‘3’, the plurality of second coordinates constituting the second surface 431 may also be moved by ‘3’ in the corresponding one direction. In the above-described example, specific numerical descriptions of the movement distance of each surface are merely examples for helping the understanding of the present disclosure, but the present disclosure is not limited thereto.

More specifically, the server 100 may match each of the plurality of first coordinates with each of the plurality of second coordinates. For example, each of the plurality of first coordinates constituting the first surface 432 and each of the plurality of second coordinates constituting the second surface 431 may be matched with each other based on a relative position. For a more specific example, the server 100 may match each of the plurality of first coordinates with each of the plurality of second coordinates parallel to each coordinate. That is, the coordinates matched with each other may be coordinates located at the shortest distance on the straight line. The server 100 may identify the movement distance of each of the plurality of first coordinates. In addition, the server 100 may move each of the plurality of second coordinates matched with each of the plurality of first coordinates based on the movement distance of each of the plurality of first coordinates.

That is, the server 100 moves each of the second coordinates matched with each first coordinate based on the movement distance of each of the plurality of first coordinates constituting the first surface, so that the server can move the second surface 431.

Accordingly, in response to the electrode model 430 being in contact with the one surface of the head shape model 330, the first surface 432 and the second surface 431 may be equally moved. In other words, the second coordinates corresponding to the respective first coordinates are moved by the distance moved by the first coordinates constituting the first surface 432 of the electrode model 430, so that the second surface 431 is moved as much as the first surface 432 is moved.

Through the above-described process, the server 100 can perform the electrode attachment simulation on the process in which the electrode model 430 is contacted or attached to the head shape model 330. The visualization information associated with how to attach the electrode patch to the actual scalp area of the user (that is, the optimal stimulation position) can be provided through the electrode attachment simulation. That is, the user can acquire the visualization information associated with medical assistance through the electrode attachment simulation.

According to one embodiment of the present invention, the server 100 may perform the candidate electrode attachment simulation to attach the candidate electrode model 430 based on the electrode model 430 that is in contact with the head shape model 330. The candidate electrode model 430 may have a property of being attached within a predetermined separation distance from the electrode model 430 on the head shape model 330. For example, in the case of transcranial direct current stimulation, according to the size and direction of the stimulation to be applied, additional electrode patches (that is, candidate electrodes) may be arranged in addition to the electrode patches attached in advance to the scalp. In this case, the additional electrode patches need to be arranged in the same direction within a limited radius as the electrode patch attached to the head shape. For example, it may be difficult to arrange each electrode patch in the same direction on the scalp, which is an irregular surface. Specifically, in the case of a two-dimensional coordinate associated with x and y, the degree of 0 may be easily defined as a direction parallel to a specific axis, but in the case of a three-dimensional coordinate associated with x, y, and z, the degree of 0 may not be easily defined based on a specific axis. In other words, as the scalp surface is bumpy, the direction may be changed according to a slight change in position, so that it may be difficult to attach an additional electrode patch on the scalp surface in a consistent direction with the electrode patch.

In one embodiment of the present invention, the server 100 may locate the candidate electrode model 430 based on the attachment position of the electrode model 430. That is, the server 100 may locate the candidate electrode model 430 in association with the attachment position of the electrode model 430 on the head shape model 330. For example, the server 100 may locate the candidate electrode model 430 within a preset interval from the electrode model 430.

In one embodiment, the server 100 may determine the attachment orientation of the candidate electrode model 430 based on the attachment form of the electrode model 430. For a specific example, as illustrated in FIG. 9, the electrode model 430 may be implemented as a hexahedron having a thin thickness. The server 100 can determine the attachment direction of the candidate electrode model 430 based on the direction in which the longest side of the corresponding electrode model 430 is formed in the electrode model 430 attached to the head shape model 330. In other words, the attachment direction of the candidate electrode model 430 may be determined so that the longest side of the electrode model 430 and the long side of the candidate electrode model 430 are close to parallel. In the above description, the case in which the first surface and the second surface are rectangular is specifically and exemplarily described, but the present disclosure is not limited thereto.

That is, the server 100 identifies the attachment direction of the electrode model 430 on the head shape model 330 and attaches the candidate electrode model 430 based on the identified attachment direction so that the candidate electrode model may be arranged in a uniform direction with the electrode model 430.

According to another embodiment of the present invention, the server 100 may sequentially change the orientation angle of the candidate electrode model 430 and may acquire the plurality of direction vectors according to the change in orientation angle. Herein, when the candidate electrode model 430 is in contact with the surface of the head shape model 330, the orientation angle is in association with rotation with the one point of the head shape model 330 in contact with the candidate electrode model 430 as a central axis. As a specific example, the server 100 may change the orientation angle of the candidate electrode model 430 as illustrated in FIG. 11. For example, as the server 100 changes the orientation angle of the candidate electrode model 430, as illustrated in FIG. 11 (b), the candidate electrode model can be rotated (430a-1) on the surface of the head shape model 330.

That is, the server 100 may sequentially change the orientation angle of the candidate electrode model 430 from the degree of 0 to the degrees of 360 and may acquire each of a plurality of direction vectors according to each angle change. For example, when the orientation angle of the candidate electrode model 430 is changed to the degree of 90, the server 100 may acquire the direction vector −1 related thereto, and when the orientation angle of the candidate electrode model 430 is changed to the degree of 180, the server 100 may acquire the direction vector −2 related thereto. In this case, the direction vector −1 and the direction vector −2 may be different from each other.

In addition, the server 100 may determine the attachment direction of the candidate electrode model 430 on the head shape model 330 based on the comparison of the acquired plurality of direction vectors and the first direction vector of the electrode model 430. Specifically, the server 100 may sequentially change the orientation angle from degree of 0 to the degree of 360, may acquire each of the plurality of direction vectors according to each angle change, and may identify the vector most similar to the first direction vector of the electrode model 430 among the plurality of direction vectors. In addition, the server 100 may determine the attachment direction of the candidate electrode model 430 based on the orientation angle corresponding to the vector most similar to the first direction vector of the electrode model 430.

That is, the server 100 may acquire a plurality of direction vectors associated with the candidate electrode model 430 through the change in orientation angle and may determine the attachment direction of the candidate electrode model 430 based on the orientation angle having the direction vector most similar to the first direction vector of the electrode model 430 among the plurality of direction vectors. Accordingly, the candidate electrode model 430 may be arranged in a uniform direction with the electrode model 430. Through the above-described process, the server 100 can arrange the candidate electrode model 430 with the electrode model 430 at a uniform interval and in a uniform direction on the uneven scalp surface.

According to one embodiment of the present invention, optimal stimulation position information may include one or more optimal stimulation positions sub-information. For example, one or more optimal stimulation positions sub-information may be output through the optimal position determination model. That is, according to the embodiment, there may be several positions on the scalp that maximize the electrical stimulation applied to the stimulation target point. In other words, when stimulation is applied with one or more electrodes attached to each of the one or more optimal stimulation positions sub-information, the efficiency of stimulation delivery can be further improved.

To this end, the server 100 may perform one or more electrode attachment simulations on the head shape model 330 in response to each of one or more optimal stimulation positions sub-information. Each electrode attachment simulation may be to provide information on the process in which each electrode model is contacted or attached to the head shape model 330 in response to each optimal stimulation position sub-information. For example, the visualization information associated with how to attach each electrode patch on several areas of the actual scalp of the user can be provided through each electrode attachment simulation.

According to one embodiment, the server 100 may identify whether at least one electrode model 430 overlaps on the head shape model 330 as a result of one or more electrode attachment simulations. For example, according to the size of each arrangement of the electrode model or one or more optimal stimulation positions sub-information, one or more electrode models may be superimposed on the head shape model 330. In addition, the server 100 may modify at least one optimal stimulation position sub-information based on the identified overlapped electrode model. In one embodiment, the server 100 may modify the provided shape of at least one electrode model based on the identified overlapped electrode model.

In other words, due to the influence of the arrangement size of each electrode model or the distance between optimal positions, overlapped electrode models may occur in one or more electrode attachment simulation processes. In this case, the server 100 may modify the optimal stimulation position sub-information so that at least one electrode model among the overlapped electrode models is arranged in a different position. In addition, the server 100 may change the shape of at least one electrode model among the overlapped electrode models (for example, reduce the size or change the shape so as not to overlap each other).

Through the above process, the server 100 can perform one or more electrode attachment simulations associated with the process in which one or more electrode models are attached to the head shape model 330. The visualization information associated with how to attach each of the electrode patches to various areas (that is, each of the optimal stimulation positions) on the actual scalp of the user can be provided through the electrode attachment simulation. In addition, the server 100 predicts the possibility of overlap between each electrode model. If it is expected to overlap, information on different optimal stimulation positions can be provided to avoid interference between the electrodes, or the shape of the electrode model can be changed. Accordingly, the user can acquire information associated with a countermeasure for an interference situation that may occur in the plurality of electrode patch attachment process.

The optimal stimulation position calculation method using the aforementioned AI model was described with reference to the flowchart illustrated in the figure. For a simple explanation, the optimal stimulation position calculation method using the AI model has been described by illustrating a series of blocks, but the present invention is not limited to the order of the blocks, and some blocks are in an order different from that illustrated and operated in this specification It can be performed with or simultaneously. In addition, new blocks not described in this specification and drawings may be added, or some blocks may be deleted or changed.

Above, although the embodiment of the present invention has been described with reference to the accompanying drawings, those skilled in the art to which the present invention pertains will understand that the present invention can be embodied in other specific forms without changing its technical spirit or essential features. Therefore, it should be understood that the embodiments described above are illustrative in all respects and not restrictive.

1. An optimal stimulation position calculation method using an AI model, performed on a computing device, the method comprising:
generating a head shape model based on user diagnosis information;
generating a spherical model based on the head shape model;
identifying a plurality of transfer coordinate data corresponding to each of a plurality of spherical coordinate data associated with the spherical model; and
acquiring optimal stimulation position information by processing the plurality of transfer coordinate data as an input of an optimal position determination model,
wherein the plurality of transfer coordinate data are data associated with perpendicular coordinates that can be expressed in the head shape model.

2. The method according to claim 1,
wherein the user diagnosis information is information that is a basis for generating the head shape model and includes medical image information about a brain or head shape of a user,
wherein the head shape model is a three-dimensional brain map model associated with a brain or head shape of an actual user,
wherein the spherical model is implemented through at least a portion of a three-dimensional sphere model having a specific radius, and
wherein the plurality of spherical coordinate data are data associated with spherical coordinates that can be expressed in the spherical model.

3. The method according to claim 1,
wherein the generating the head shape model based on the user diagnosis information includes:
acquiring one or more brain area images by processing the user diagnosis information as an input of an image area classification model;
performing pre-processing on the one or more brain area images; and
generating the head shape model based on the pre-processed one or more brain area images, and
wherein the image area classification model is a neural network model based on a convolutional neural network (CNN), it is to be learned through learning data including learning input data associated with a plurality of the user diagnosis information and learning output data associated with brain area classification information corresponding to each of the user diagnosis information.

4. The method according to claim 3, wherein the generating the spherical model based on the user diagnosis information includes:
developing one or more spheres based on a center point of the head shape model;
identifying a first sphere among the developed one or more spheres having a minimized distance between a surface of a sphere and a surface of the head shape model and having a maximum radius; and
generating the spherical model based on the identified first sphere.

5. The method according to claim 1, wherein the optimal position determination model derives brain activity prediction information corresponding to each of the plurality of transfer coordinate data, identifies optimal transfer coordinate data in which a loss function associated with a difference between each of the derived brain activity prediction information and each of reference brain activity information corresponding to each of the plurality of transfer coordinate data is smaller than or equal to a preset reference value, and outputs the optimal stimulation position information based on the identified optimal transfer coordinate data,

6. The method according to claim 1, wherein the method includes:
determining whether or not at least two or more spherical coordinate data among the plurality of spherical coordinate data indicate one point on the head shape model in an overlapped manner; and
performing correction on a portion of the at least two or more spherical coordinate data when the at least two or more spherical coordinate data indicate one point on the head shape model in an overlapped manner.

7. The method according to claim 6, wherein the performing the correction on a portion of the at least two or more spherical coordinate data includes:
removing at least one spherical coordinate data among the at least two or more spherical coordinate data indicating the one point on the head shape model in an overlapped manner; or
correcting coordinates of the at least one spherical coordinate data among at least two or more spherical coordinate data indicating the one point on the head shape model in an overlapped manner.

8. The method according to claim 1, wherein the method further includes:
arranging an electrode model at a first position corresponding to the optimal stimulation position information; and
performing electrode attachment simulation on the head shape model by gradually moving the electrode model located at the first position to a position corresponding to the optimal stimulation position information, and
wherein the first position is one position in a normal vector direction of the optimal stimulation position information.

9. A server calculating an optimal stimulation position by using an AI model, the server comprising:
a processor;
a network interface;
a memory; and
a computer program loaded on the memory and executed by the processor,
wherein the computer program includes:
an instruction for generating a head shape model based on user diagnosis information;
an instruction for generating a spherical model based on the head shape model;
an instruction for identifying a plurality of transfer coordinate data corresponding to each of the plurality of spherical coordinate data associated with the spherical model; and
an instruction for acquiring optimal stimulation position information by processing the plurality of transfer coordinate data as an input of an optimal position determination model, and
wherein the plurality of transfer coordinate data are data associated with perpendicular coordinates that can be expressed in the head shape model.

10. A computer program recorded on a computer-readable recording medium,
combined with a computing device, and
causing the computing device to execute:
generating a head shape model based on user diagnosis information;
generating a spherical model based on the head shape model;
identifying a plurality of transfer coordinate data corresponding to each of a plurality of spherical coordinate data associated with the spherical model, wherein the plurality of transfer coordinate data is data associated with perpendicular coordinates that can be expressed in the head shape model; and
acquiring optimal stimulation position information by processing the plurality of transfer coordinate data as an input of an optimal position determination model.
